(19) United States
(12) Patent Application Publication
(10) Pub. No.: US 2020/0406658 A1
(43) Pub. Date: Dec. 31, 2020

(54) THERMOSENSITIVE RECORDING MEDIUM

(71) Applicant: Nippon Paper Industries Co., Ltd., Tokyo (JP)

(72) Inventors: Kenji Hirai, Tokyo (JP); Koji Kutsuwa, Tokyo (JP); Yoshimi Midorikawa, Tokyo (JP)

(21) Appl. No.: 16/978,626

(22) PCT Filed: Mar. 1, 2019
(86) PCT No.: PCT/JP2019/007989

§ 371 Date: Sep. 4, 2020

(30) Foreign Application Priority Data
Mar. 5, 2018 (JP) 2018038244

(51) Int. Cl. B41M 5/333; C07C 317/50

(52) U.S. Cl. CPC B41M 5/3336; C07C 317/50

(57) ABSTRACT

Provided is a thermosensitive recording medium, which is excellent in color developing property, especially excellent in bar code readability, under severe environment, further, excellent in resistance to discoloration under severe environment. That is a thermosensitive recording medium having a thermosensitive recording layer comprising a colorless or pale colored electron donating leuco dye and an electron accepting color developing agent on a substrate, wherein the thermosensitive recording layer comprises, as the electron accepting color developing agent, a specific sulfone compound and a phenolic compound in a specific ratio, wherein the phenolic compound contains two or more hydroxyl groups and two or more diphenylsulfone backbone but contains neither urethane bond nor urea bond. The thermosensitive recording layer optionally may further comprise a specific urea-urethane-based compound.

### FIELD OF THE INVENTION

The present invention relates to a thermosensitive recording medium for recording image by utilizing a coloring reaction between a colorless or pale colored electron donating leuco dye (referred to as “leuco dye”) and an electron accepting color developing agent (referred to as “color developing agent”), which is excellent in color developing property, especially excellent in bar code readability, under severe environment, further, excellent in resistance to discoloration under severe environment.

### BACKGROUND OF THE INVENTION

Thermosensitive recording media are ordinarily prepared by mixing together a leuco dye and a color developing agent, such as a phenolic compound and the like, after grinding them into fine particles, preparing a coating solution by adding a binder, a filler, a sensitizer, a slipping agent and other aids to the mixture and applying the coating solution onto a substrate such as paper, synthetic paper, film, plastic and the like. Thermosensitive recording medium develops color through an instantaneous chemical reaction when heated by a thermal head, hot stamp, hot pen, laser light or the like to yield a recorded image. Such thermosensitive recording media are used extensively in recording media such as facsimile devices, computer terminal printers, automatic ticket dispensers, recorders for meters, receipts at super markets and convenience stores and the like.

In recent years, the use of the thermosensitive recording medium is expanding, such as various ticket, receipts, labels, ATM of Bank, meter reading of gas and electricity, cash vouchers, such as car racing or horseracing betting. Then the thermosensitive recording medium has been required to have a good preservation property of an image portion and a blank portion under severe environment such as high temperature condition like in a car in mid-summer, or to have a plasticizer resistance that does not cause problems in the readability of the printed portion even when the thermosensitive recording medium is stored in contact with a film or synthetic leather for a long period of time.

Therefore, thermosensitive recording media using a combination of specific color developing agent and a stabilizer to improve storability of image portion (Reference 1), thermosensitive recording media using a combination of two specific types of color developing agents to improve color developing property and image storage stability (Reference 2), thermosensitive recording media using a combination of two types of color developing agents such as a phenol compound and a BPS based color developing agent to improve storage stability (References 3 and 4), thermosensitive recording medium using a combination of two types of sulfone-based color developing agents (Reference 5), etc. are disclosed.

Furthermore, the present inventors disclosed a thermosensitive recording medium which is excellent in color developing property, particularly in bar code readability, under severe environment, by using a combination of two types of color developing agents, a urea-urethane-based compound and a BPS based compound (Reference 6).

### REFERENCES

### Reference 1: Japanese Patent Application Public Disclosure No. 2001-347757

### Reference 2: Japanese Patent Application Public Disclosure No. 2006-264255

### Reference 3: Japanese Patent Application Public Disclosure No. 2000-135863

### Reference 4: Japanese Patent Application Public Disclosure No. 2000-135868

### Reference 5: Japanese Patent Application Public Disclosure No. 2019-001141

### Reference 6: International Publication WO2016/204215

### Problems to be Solved by the Invention

The storability of image area and blank area of a thermosensitive recording medium is important, when used for the above-mentioned applications, such as ticket, labels and the like under severe environment, which have been increasingly used in recent years. For example, when the thermosensitive recording medium or the like using a combination of two specific types of color developing agents (Reference 2) is used under severe environment, the blank part develops color then the barcode readability becomes insufficient, since the heat resistance of the blank part is poor (see Comparative Examples 1, 2, 4, and 6 below).

Then, the present inventors developed a thermosensitive recording medium which is excellent in color developing property, particularly in bar code readability, under severe environment, by using a combination of two types of color developing agents, a urea-urethane-based compound and a BPS based compound (Reference 6).

However, as a result of market research, it was found that the thermosensitive recording medium (Reference 6) further needs to improve the resistance to discoloration under severe environment (see Comparative Example 3 below).

Therefore, the object of the present invention is to provide a thermosensitive recording medium, which is excellent in color developing property, especially excellent in bar code readability, under severe environment, further, excellent in resistance to discoloration under severe environment.

In the present invention, the severe environment or severe condition means, for example, high temperature and/or high humidity conditions, the high temperature means, for example, 70 degree C. or higher, and the high humidity means, for example, 80% RH or higher. Further, the severe conditions include heating in a microwave oven (for example, heating with energy of about 500 W to 1500 W for about 1 to 5 minutes) and the like.

For example, in the case of using a conventional thermosensitive recording medium for a food label, when the food on which the food label after printing is attached is heated in a microwave oven, the white part of the food label is colored (i.e. discolored), the aesthetic appearance is impaired and it becomes difficult to read the printed information on the food label (see Comparative Examples 1 to 4 and 6 below).

Furthermore, the importance of plasticizer resistance, which does not cause a problem in the readability of the printed area even when stored in contact with film or synthetic leather for a long time, is becoming more important.

### Means to Solve the Problems

As a result of intensive studies, the present inventors have found that the above problems can be solved by using the thermosensitive recording layer comprising, as the electron accepting color developing agent, a specific sulfone compound and a phenolic compound in a specific ratio, wherein the phenolic compound contains two or more hydroxyl groups and two or more diphenylsulfone backbone but contains neither urethane bond nor urea bond, and then completed the present invention.

That is, the present invention provides a thermosensitive recording medium having a thermosensitive recording layer comprising a colorless or pale colored electron donating leuco dye and an electron accepting color developing agent on a substrate, wherein the thermosensitive recording layer comprises a sulfone compound and a phenolic compound as the electron accepting color developing agent, wherein the sulfone compound is represented by the general formula 1 and the phenolic compound is a compound containing two or more hydroxyl groups and two or more diphenylsulfone backbone and containing neither urethane bond nor urea bond, wherein the content of the phenolic compound is from 0.01 to 1.0 parts by weight per 1 part by weight of the sulfone compound.

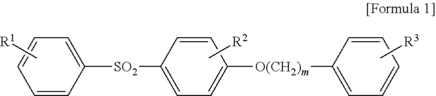

wherein R¹ represents a hydrogen atom or a hydroxyl group, R² and R³, which may be identical to or different from the others, represent a hydrogen atom or an alkyl group or an alkoxy group, having 1-6 carbon atoms, and m represents an integer of 1 to 3.

In order to further improve the plasticizer resistance, the thermosensitive recording layer may further comprise, as the electron accepting color developing agent, an urea-urethane-based compound represented by the following general formula:

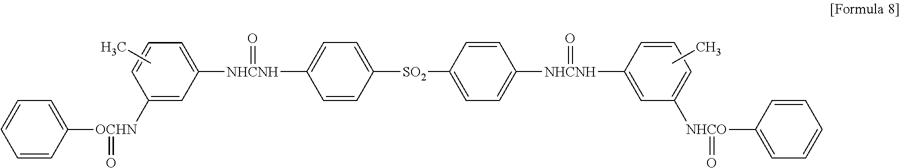

### Advantages of the Present Invention

According to the present invention, provided is a thermosensitive recording medium, which is excellent in color developing property, especially excellent in bar code readability, under severe environment, further, excellent in resistance to discoloration under severe environment.

Furthermore, by using the three kinds of color developing agents including the urea-urethane-based compound, the resistance to discoloration can be improved.

### DETAILED DESCRIPTION OF THE INVENTION

The thermosensitive recording medium of the present invention comprises a thermosensitive recording layer comprising a colorless or pale colored electron donating leuco dye and an electron accepting color developing agent on a substrate, wherein the thermosensitive recording layer comprises, as the electron accepting color developing agent, a specific sulfone compound and a phenolic compound in a specific ratio.

The sulfone compound used in the present invention is represented by the general formula 1 (Formula 1).

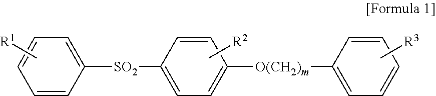

In the above formula (Formula 1), R¹ represents a hydrogen atom or a hydroxyl group, preferably a hydroxyl group.

R² and R³, which may be identical to or different from the others, represent a hydrogen atom or an alkyl group or an alkoxy group, in which the alkyl group or the alkoxy group has 1-6, preferably 1-3 carbon atoms. More preferably, at least one of R² and R³ represents a hydrogen atom, further preferably, both of R² and R³ are hydrogen atoms.

m represents an integer of 1 to 3, preferably 1 or 2, more preferably 1.

As this alkyl group, linear or branched alkyl groups having 1 to 6 carbon atoms are mentioned, which include, for example, methyl group, ethyl group, propyl group, isopropyl group, butyl group, isobutyl group, sec-butyl group, tert-butyl group, pentyl group, isopentyl group, neopentyl group, tert-pentyl group, 1-methylbutyl group, 2-methylbutyl group, 1,2-dimethylpropyl group, hexyl group, isohexyl group, 1-methylpentyl group, 2-methylpentyl group, 3-methylpentyl group, 1,1-dimethylbutyl group, 1,2-dimethylbutyl group, 2,2-dimethylbutyl group, 1,3-dimethylbutyl group, 2,3-dimethylbutyl group, 3,3-dimethylbutyl group, 1-ethylbutyl group, 2-ethylbutyl group, 1,1,2-trimethylpropyl group, 1,2,2-trimethylpropyl group, 1-ethyl-1-methylpropyl group, 1-ethyl-2-methylpropyl group and the like. The alkyl group is preferably an alkyl group having 1 to 3 carbon atoms, such as methyl group, ethyl group, propyl group and isopropyl group.

As this alkoxy group, linear or branched alkoxy groups having 1 to 6 carbon atoms are mentioned, which include, for example, methoxy group, ethoxy group, propoxy group, isopropoxy group, butoxy group, isobutoxy group, sec-butoxy group, tert-butoxy group, pentyloxy (amyloxy) group, isopentyloxy group, tert-pentyloxy group, neopentyloxy group, 2-methylbutoxy group, 1,2-dimethylpropoxy group, 1-ethylpropoxy group, hexyloxy group. The alkoxy group is preferably methoxy group, ethoxy group, propoxy group, or isopropoxy group.

The preferable sulfone compound used in the present invention is represented by the general formula (Formula 5).

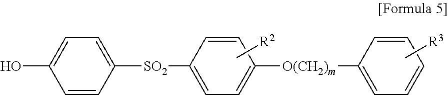

wherein R², R³ and m are as defined above.

More preferable sulfone compound used in the present invention includes 4-hydroxy-4′-benzyloxydiphenylsulfone, 4-hydroxy-4′-phenethyloxydiphenylsulfone, 4-hydroxy-4′-(3-phenylpropoxy) diphenylsulfone, most preferably 4-hydroxy-4′-benzyloxydiphenylsulfone (represented by the following formula (Formula 6)).

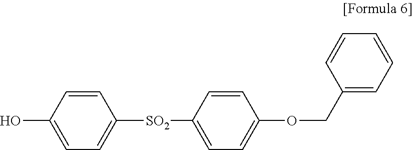

The phenolic compound used in the present invention contains 2 or more, preferably 2 to 4, more preferably 2 hydroxyl groups and 2 or more, preferably 2 to 12 diphenylsulfone backbones and contains neither urethane bond nor urea bond.

As such a phenolic compound, a cross-linking compound represented by the following general formula (Formula 2) is preferable.

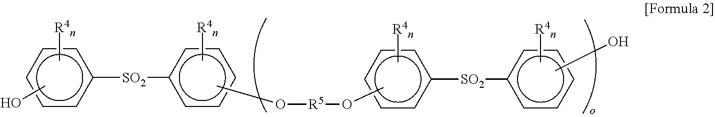

In the formula (formula 2), R⁴, which may be identical or different, preferably identical, represents a halogen atom, or an alkyl group or an alkenyl group having 1 to 6 carbon atoms.

This alkyl group or alkenyl group is an alkyl or alkenyl group having 1-6 carbon atoms, for example, methyl, ethyl, n-propyl, isopropyl, n-butyl, sec-butyl, t-butyl, n-pentyl, isopentyl, neopentyl, t-pentyl, n-hexyl, iso-hexyl, 1-methylpentyl, 2-methylpentyl, vinyl, allyl, isopropenyl, 1-propenyl, 2-butenyl, 3-butenyl, 1,3-butadienyl, 2-methyl-2-propenyl and the like.

The halogen atom includes chlorine atom, bromine atom, fluorine atom or iodine atom, preferably represents chlorine atom or bromine atom.

n, which may be identical or different, preferably identical, represents an integer of from 0 to 4, preferably 0.

OH group and —OR⁵O— group preferably locate in a para-position to SO₂ group.

o is an integer of from 1 to 11.

This compound is preferably a mixture of those in which p are from 1 to 11.

R⁵, which may be identical or different, is preferably identical.

R⁵ may be a hydrocarbon group having 1 to 12, preferably 3 to 7 carbon atoms, which may have an ether bond, which may be saturated or unsaturated, preferably saturated, which may be linear or branched, preferably linear. Such hydrocarbons includes a polyalkylene oxide chain or an alkylene group, preferably is a polyalkylene oxide chain. When R^{b} is a polyalkylene oxide chain, —OR⁵O— includes —O—(CₚH₂ₚO)₁₋₃— in which p is 2 to 4, preferably 2 to 3, more preferably 2. The alkylene group includes —C_{q}H_{2q}—, in which q is 1 to 12, preferably 3 to 7.

R⁵ may be a substituted phenylene group represented by a general formula (formula 3) below:

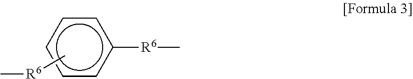

In the formula, R⁶ represents a methylene group or ethylene group. Two R⁶ preferably are in a para position each other.

In addition, R⁵ may be a divalent group represented by a general formula (formula 4) below:

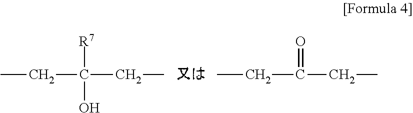

In the formula, R⁷ represents a hydrogen atom or an alkyl group having 1 to 4 carbon atoms, preferably a hydrogen atom.

Among these, R⁵ preferably is a hydrocarbon group having 1 to 12 carbon atoms, which may have an ether bond, which may be saturated or unsaturated, which may be linear or branched, as described above.

As such a diphenyl sulfone cross-link type compound represented by the above formula 2, a compound represented by the formula below (formula 7, D-90 manufactured by Nippon Soda Co., Ltd.) is preferred.

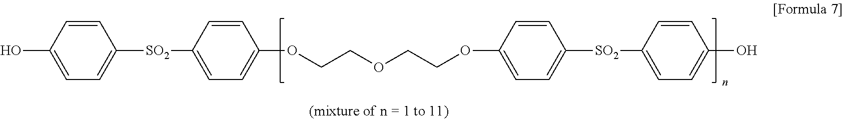

The thermosensitive recording layer of the present invention preferably contains, as a developer, further an urea-urethane-based compound represented by the following general formula (Formula 8), in addition to the specific sulfone compound and the specific phenolic compound.

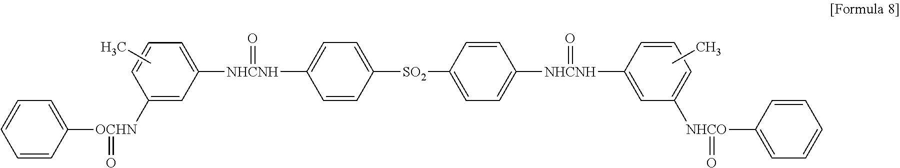

The urea-urethane-based compound, used as the color developing agent in the present invention, includes specifically three kinds of compounds represented by the following formulas, Formula 10 to 12. These may be used individually or as a mixture of at least two of them.

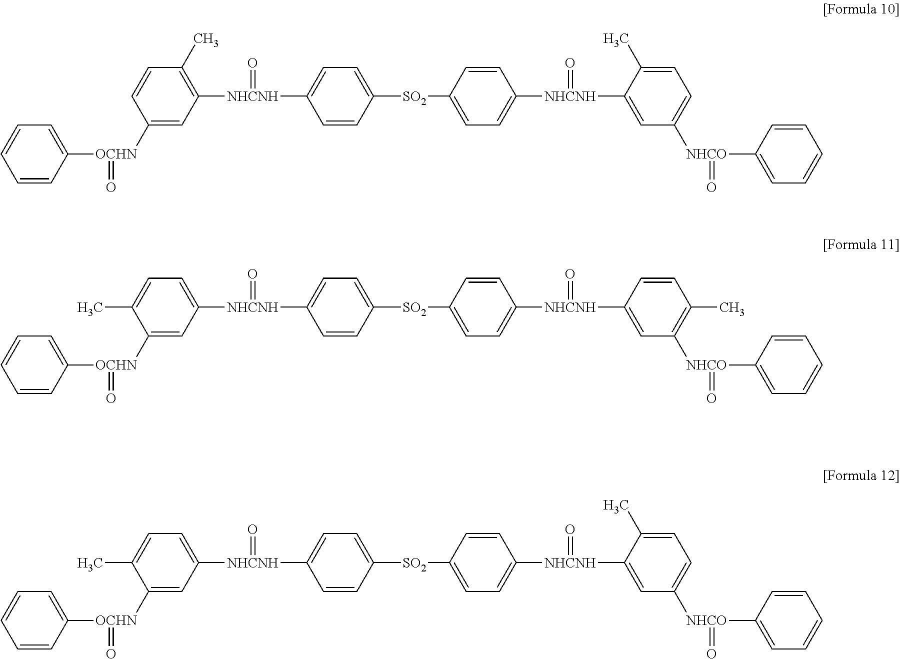

In the present invention, the thermosensitive recording layer of the present invention contains, as the electron accepting color developing agent, from 0.01 to 1.0 parts by weight of the phenolic compound per 1 part by weight of the sulfone compound. Further, the thermosensitive recording layer contains the above phenolic compound in an amount of preferably 0.05 part by weight or more, more preferably 0.1 part by weight or more, per 1 part by weight of the sulfone compound. And the thermosensitive recording layer contains the above phenolic compound in an amount of preferably 0.8 part by weight or less, more preferably 0.5 part by weight or less, further preferably less than 0.5 part by weight, per 1 part by weight of the sulfone compound.

When the thermosensitive recording layer of the present invention further contains an urea-urethane-based compound as the electron accepting color developing agent, the thermosensitive recording layer contains from 0.01 to 1.0 parts by weight of the urea-urethane-based compound per 1 part by weight of the sulfone compound. Further, the thermosensitive recording layer contains the above urea-urethane-based compound in an amount of preferably 0.05 part by weight or more, more preferably 0.1 part by weight or more, per 1 part by weight of the sulfone compound. And the thermosensitive recording layer contains the above urea-urethane-based compound in an amount of preferably 0.8 part by weight or less, more preferably 0.6 part by weight or less, further preferably less than 0.6 part by weight, per 1 part by weight of the sulfone compound.

As the ratio of the phenolic compound to the sulfone compound is within this range, the thermosensitive recording medium becomes excellent in color developing property, especially excellent in bar code readability, under severe environment, further, excellent in resistance to discoloration under severe environment (see Examples below). When the content of the phenolic compound is less than 0.01 parts by weight per 1 part by weight of the sulfone compound, the bar code readability tends to be insufficient. And when the content of the phenolic compound exceeds 1.0 parts by weight per 1 part by weight of the sulfone compound, the suppression of discoloration may be insufficient.

In the case that the thermosensitive recording layer of the present invention further contains an urea-urethane-based compound as the electron accepting color developing agent, when the content of the urea-urethane-based compound is less than 0.01 parts by weight per 1 part by weight of the sulfone compound, the plasticizer resistance tends to be insufficient. And when the content of the urea-urethane-based compound exceeds 1.0 parts by weight per 1 part by weight of the sulfone compound, the whiteness of the white part may become deteriorated.

The thermosensitive recording layer of the present invention may contain color developing agents other than the sulfone compound, the phenolic compound and the urea-urethane-based compound. In this case, the total amount of the sulfone compound and the phenolic compound and the urea-urethane-based compound which is optionally contained is preferably 50% by weight or more, more preferably 70% by weight or more, further preferably 90% by weight or more, and particularly preferably 100% by weight (which means that all of the color developing agents contained in the thermosensitive recording layer are the sulfone compound and the phenolic compound and the urea-urethane-based compound which is optionally contained) of the total amount of the color developing agents contained in the thermosensitive recording layer, which contains the sulfone compound and the phenolic compound and the urea-urethane-based compound which is optionally contained.

As the color developing agents other than the sulfone compound, the phenolic compound and the urea-urethane-based compound, for example, activated clay, attapulgite, colloidal silica, inorganic acidic substances such as aluminum silicate and the like, 4,4′-isopropylidene diphenol, 1,1-bis(4-hydroxyphenyl) cyclohexane, 2,2-bis(4-hydroxyphenyl)-4-methylpentane, 4,4′-dihydroxydiphenyl sulfide, hydroquinone monobenzyl ether, benzyl 4-hydroxybenzoate, 4,4′-dihydroxy diphenyl sulfone, 2,4′-dihydroxy diphenyl sulfone, 4-hydroxy-4′-isopropoxy diphenyl sulfone, 4-hydroxy-4′-n-propoxy diphenyl sulfone, 4-hydroxy-4′-allyloxy diphenyl sulfone, bis(3-allyl-4-hydroxyphenyl) sulfone, 4-hydroxy-4′-methyldiphenyl sulfone, 3,4-dihydroxyphenyl-4′-methyl phenyl sulfone, 1-[4-(4-hydroxyphenyl-sulfonyl) phenoxy]-4-[4-(4-isopropoxyphenyl sulfonyl) phenoxy] butane, phenol condensate composition described in Japanese Patent Application Public Disclosure No. 2003-154760, aminobenzene sulfonamide derivatives described in Japanese Patent Application Public Disclosure No. H08-59603, bis(4-hydroxyphenyl thioethoxy) methane, 1,5-di(4-hydroxyphenyl thio)-3-oxapentane, butyl bis(p-hydroxyphenyl) acetate, methyl bis(p-hydroxyphenyl) acetate, 1,1-bis(4-hydroxyphenyl)-1-phenylethane, 1,4-bis[α-methyl-α-(4′-hydroxyphenyl)ethyl] benzene, 1,3-bis[α-methyl-α-(4′-hydroxyphenyl)ethyl] benzene, di(4-hydroxy-3-methylphenyl) sulfide, 2,2′-thiobis(3-tert-octylphenol), 2,2′-thiobis(4-tert-octylphenol), compounds described in International Publication WO02/081229 or Japanese Patent Application Public Disclosure No. 2002-301873, thiourea compounds such as N,N′-di-m-chlorophenyl thiourea and the like, p-chlorobenzoic acid, stearyl gallate, bis[zinc 4-octyloxy carbonylamino salicylate] dihydrate, 4-[2-(p-methoxyphenoxy) ethyloxy] salicylic acid, 4-[3-(p-tolylsulfonyl) propyloxy] salicylic acid, aromatic carboxylic acids such as 5-[p-(2-p-methoxyphenoxyethoxy) cumyl] salicylic acid, and salts of these aromatic carboxylic acids and polyvalent metals such as zinc, magnesium, aluminum, calcium, titanium, manganese, tin, nickel and the like, and, furthermore, antipirin complexes of zinc thiocyanate and complex zinc salts of terephthal aldehyde acid with other aromatic carboxylic acids and the like may be cited.

These color developing agents may be used individually or as a mixture of at least two of them.

1-[4-(4-hydroxyphenyl-sulfonyl) phenoxy]-4-[4-(4-isopropoxyphenyl sulfonyl) phenoxy] butane is available, for example, under the trade name of TOMILAC 214 produced by Mitsubishi Chemical Corporation. The phenol condensate composition described in Japanese Patent Application Public Disclosure No. 2003-154760 is available, for example, under the trade name of J TOMILAC 224 produced by Mitsubishi Chemical Corporation. The compound described in International Publication WO02/081229 is available, for example, under the trade names of NKK-395 and D-100 produced by Nippon Soda Co., Ltd. In addition, high molecular weight aliphatic acid metal complex salts described in Japanese Patent Application Public Disclosure No. H10-258577 and metal chelate type color developing components such as polyvalent hydroxy aromatic compounds and the like may also be present.

The various materials used in the thermosensitive recording layer of the thermosensitive recording medium of the present invention are shown below. These materials, such as binders, crosslinking agents, pigments and the like, may be used also for other coating layer(s) other than the thermosensitive recording layer, such as the protective layer and the undercoat layer, unless the desired effects for the problems described above are not hampered.

All of the leuco dyes well known in the conventional field of pressure sensitive and thermosensitive recording media may be used as the electron donating leuco dye in the present invention. Although the leuco dye is not particularly restricted, triphenylmethane type compounds, fluorane type compounds, fluorene type compounds, divinyl type compounds and the like are preferred as the leuco dye. Specific examples of the typical colorless to pale colored basic colorless leuco dye (leuco dye precursors) are shown below. In addition, these leuco dye precursors may be used individually and also in mixtures of at least two of them.

### <Triphenylmethane Type Leuco Dyes>

3,3-bis(p-Dimethyl aminophenyl)-6-dimethylaminophthalide [alternate name: crystal violet lactone] and 3,3-bis(p-Dimethyl aminophenyl) phthalide [alternate name: malachite green lactone]

### <Fluorane Type Leuco Dyes>

3-Diethylamino-6-methylfluorane, 3-diethylamino-6-methyl-7-anilinofluorane, 3-diethylamino-6-methyl-7-(o,p-dimethylanilino)fluorane, 3-diethylamino-6-methyl-7-chlorofluoran, 3-diethylamino-6-methyl-7-(m-trifluoromethylanilino) fluorane, 3-diethylamino-6-methyl-7-(o-chloroanilino) fluorane, 3-diethylamino-6-methyl-7-(p-chloroanilino) fluorane, 3-diethylamino-6-methyl-7-(o-fluoroanilino) fluorane, 3-diethylamino-6-methyl-7-(m-methylanilino) fluorane, 3-diethylamino-6-methyl-7-n-octylanilino fluorane, 3-diethylamino-6-methyl-7-n-octylamino fluorane, 3-diethylamino-6-methyl-7-benzylamino fluorane, 3-diethylamino-6-methyl-7-dibenzylamino fluorane, 3-diethylamino-6-chloro-7-methyl fluorane, 3-diethylamino-6-chloro-7-anilino fluorane, 3-diethylamino-6-chloro-7-p-methylanilino fluorane, 3-diethylamino-6-ethoxyethyl-7-anilino fluorane, 3-diethylamino-7-methyl fluorane, 3-diethylamino-7-chloro fluorane, 3-diethylamino-7-(m-trifluoromethylanilino)fluorane, 3-diethylamino-7-(o-chloroanilino) fluorane, 3-diethylamino-7-(p-chloroanilino)fluorane, 3-diethylamino-7-(o-fluoroanilino) fluorane, 3-diethylamino-benz[a] fluorane, 3-diethylamino-benz[c] fluorane, 3-dibutylamino-6-methyl-fluorane, 3-dibutylamino-6-methyl-7-anilino fluorane, 3-dibutylamino-6-methyl-7-(o,p-dimethylanilino) fluorane, 3-dibutylamino-7-(o-chloroanilino) fluorane, 3-butylamino-6-methyl-7-(p-chloroanilino) fluorane, 3-dibutylamino-6-methyl-7-(o-fluoroanilino) fluorane, 3-dibutylamino-6-methyl-7-(m-fluoroanilino) fluorane, 3-dibutylamino-6-methyl-chloro fluorane, 3-dibutylamino-6-ethoxyethyl-7-anilino fluorane, 3-dibutylamino-6-chloro-7-anilino fluorane, 3-dibutylamino-6-methyl-7-p-methylanilino fluorane, 3-dibutylamino-7-(o-chloroanilino) fluorane, 3-dibutylamino-7-(o-fluoroanilino) fluorane, 3-di-n-pentylamino-6-methyl-7-anilino fluorane, 3-di-n-pentylamino-6-methyl-7-(p-chloroanilino) fluorane, 3-di-n-pentylamino-7-(m-trifluoromethylanilino) fluorane, 3-di-n-pentylamino-6-chloro-7-anilino fluorane, 3-di-n-pentylamino-7-(p-chloroanilino) fluorane, 3-pyrolidino-6-methyl-7-anilino fluorane, 3-piperidino-6-methyl-7-anilino fluorane, 3-(N-methyl-N-propylamino)-6-methyl-7-anilino fluorane, 3-(N-methyl-N-cyclohexylamino)-6-methyl-7-anilino fluorane, 3-(N-ethyl-N-cyclohexylamino)-6-methyl-7-anilino fluorane, 3-(N-ethyl-N-xylylamino)-6-methyl-7-(p-chloroanilino) fluorane, 3-(N-ethyl-p-toluidino)-6-methyl-7-anilino fluorane, 3-(N-ethyl-N-isoamylamino)-6-methyl-7-anilino fluorane, 3-(N-ethyl-N-isoamylamino)-6-chloro-7-anilino fluorane, 3-(N-ethyl-N-tetrahydrofurfurylamino)-6-methyl-7-anilino fluorane, 3-(N-ethyl-N-isobutylamino)-6-methyl-7-anilino fluorane, 3-(N-ethyl-N-ethoxypropylamino)-6-methyl-7-anilino fluorane, 3-cyclohexylamino-6-chloro fluorane, 2-(4-oxahexyl)-3-dimethylamino-6-methyl-7-anilino fluorane, 2-(4-oxahexyl)-3-diethylamino-6-methyl-7-anilino fluorane, 2-(4-oxahexyl)-3-dipropylamino-6-methyl-7-anilino fluorane, 2-methyl-6-o-(p-dimethylaminophenyl) aminoanilino fluorane, 2-methoxy-6-p-(p-dimethylaminophenyl) aminoanilino fluorane, 2-chloro-3-methyl-6-p-(p-phenylaminophenyl) aminoanilino fluorane, 2-chloro-6-p-(p-dimethylaminophenyl) aminoanilino fluorane, 2-nitro-6-p-(p-diethylaminophenyl) aminoanilino fluorane, 2-amino-6-p-(p-diethylaminophenyl) aminoanilino fluorane, 2-diethylamino-6-p-(p-diethylaminophenyl) aminoanilino fluorane, 2-phenyl-6-methyl-6-p-(p-phenylaminophenyl) aminoanilino fluorane, 2-benzyl-6-p-(p-phenylaminophenyl) aminoanilino fluorane, 2-hydroxy-6-p-(p-phenylaminophenyl)aminoanilino fluorane, 3-methyl-6-p-(p-dimethylaminophenyl) aminoanilino fluorane, 3-diethylamino-6-p-(p-diethylaminophenyl) aminoanilino fluorane, 3-diethylamino-6-p-(p-dibutylaminophenyl) aminoanilino fluorane and 2,4-dimethyl-6-[(4-dimethylamino) anilino] fluorane.

### <Fluorene Type Leuco Dye>

3,6,6-Tris(dimethylamino) spiro[fluorane-9,3′-phthalide] and 3,6,6′-tris (diethylamino) spiro[fluorane-9,3′-phthalide].

### <Divinyl Type Leuco Dyes>

3,3-bis-[2-(p-dimethylaminophenyl)-2-(p-methoxyphenyl) ethenyl]-4,5,6,7-tetrabromophthalide, 3,3-bis-[2-(p-dimethylaminophenyl)-2-(p-methoxyphenyl) ethenyl]-4,5,6,7-tetrachlorophthalide, 3,3-bis-[1,1-bis (4-pyrrolidinophenyl) ethylene-2-yl] 4,5,6,7-tetra-bromophthalide, 3,3-bis-[1-(4-methoxyphenyl)-1-(4-pyrrolidinophenyl) ethylene-2-yl]-4,5,6,7-tetrachlorophthalide

### <Others>

3-(4-Diethylamino-2-ethoxyphenyl)-3-(1-ethyl-2-methylindol-3-yl)-4-azaphthalide, 3-(4-diethylamino-2-ethoxyphenyl)-3-(1-octyl-2-methylindol-3-yl)-4-azaphthalide, 3-(4-cyclohexyl ethylamino-2-methoxyphenyl)-3-(1-ethyl-2-methylindol-3-yl)-4-azaphthalide, 3,3-bis(1-ethyl-2-methylindol-3-yl)phthalide, 3,6-bis(diethylamino)fluorane-γ-(3′-nitroanilinolactam, 3,6-bis(diethylamino)fluorane-γ-(4′-nitro) anilinolactam, 1,1-bis-[2′,2′,2″,2″-tetrakis-(p-dimethylaminophenyl-ethenyl]-2,2-dinitrilethane, 1,1-bis-[2′,2′,2″,2″-tetrakis-(p-dimethylaminophenyl)-ethenyl]-2-B-naphthoylethane, 1,1-bis-[2′,2′,2″,2″-tetrakis-(p-dimethylaminophenyl-ethenyl]-2,2-diacetylethane and bis-[2,2,2′,2′-tetrakis-(p-dimethylaminophenyl)-ethenyl]-methylmalonic acid dimethyl ester.

The previously well known sensitizers may be used as the sensitizer in the thermosensitive recording medium of the present invention. As such sensitizers, aliphatic acid amides such as stearic acid amide, palmitic acid amide and the like, ethylene bis-amide, montan acid wax, polyethylene wax, 1,2-di-(3-methylphenoxy) ethane, p-benzyl biphenyl, ß-benzyloxy naphthalene, 4-biphenyl-p-tolyl ether, m-terphenyl, 1,2-diphenoxyethane, dibenzyl oxalate, di(p-chlorobenzyl) oxalate, di(p-methylbenzyl) oxalate, dibenzyl terephthalate, benzyl p-benzyloxy benzoate, di-p-tolyl carbonate, phenyl-α-naphthyl carbonate, 1,4-diethoxynaphthalene, 1-hydroxy-2-naphthoic acid phenyl ester, o-xylene-bis-(phenyl ether), 4-(m-methyl phenoxymethyl) biphenyl, 4,4′-ethylene dioxy-bis-benzoic acid dibenzyl ester, dibenzoyloxy methane, 1,2-di(3-methylphenoxy) ethylene, bis[2-(4-methoxy-phenoxy) ethyl] ether, methyl p-nitrobenzoate, phenyl p-toluene sulfonate, o-toluenesulfonamide, p-toluenesulfonamide, and the like may be listed as examples. These sensitizers may be used individually and as mixtures of at least two of them.

As a pigment used in the the present invention, kaolin, calcined kaolin, calcium carbonate, aluminum oxide, titanium oxide, magnesium carbonate, aluminum silicate, magnesium silicate, calcium silicate, aluminum hydroxide, silica and the like may be used. These pigments may be used in combinations depending on the required quality.

As the binder used in the present invention, polyvinyl alcohols such as completely saponified polyvinyl alcohol, partially saponified polyvinyl alcohol; modified polyvinyl alcohols such as acetoacetylated polyvinyl alcohol, carboxyl-modified polyvinyl alcohol, amide-modified polyvinyl alcohol, sulfonic acid-modified polyvinyl alcohol, butyral-modified polyvinyl alcohol, olefin-modified polyvinyl alcohol, nitrile-modified polyvinyl alcohol, pyrolidone-modified polyvinyl alcohol, silicone-modified polyvinyl alcohol and the like; acrylic resins comprising (meth) acrylic acid and a monomer component copolymerizable with (meth) acrylic acid (excluding olefin); cellulose derivatives such as hydroxyethyl cellulose, methyl cellulose, ethyl cellulose, carboxymethyl cellulose, acetyl cellulose and the like; starches such as oxidized starch, etherified starch and esterified starch and the like; styrene-maleic anhydride copolymer; styrene-butadiene copolymer; casein; gum arabic; polyvinyl chloride; polyvinyl acetate; polyacrylamide; polyacrylic acid ester; polyvinyl butylal, polystyrol and their copolymers; silicone resins; petroleum resins; terpene resins; ketone resins; cumaron resins and the like may be listed as examples. The polymeric substances may be used upon dissolving them in a solvent such as water, alcohol, ketones, esters, hydrocarbons and the like or upon emulsifying or dispersing into a paste in water or other media. These polymeric materials may also be used in combinations according to the qualities demanded.

The content (in solid) of the binder in the thermosensitive recording layer is preferably about 5 to 25% by weight.

As the crosslinking agent used in the present invention, zirconium compounds such as zirconium chloride, zirconium sulfate, zirconium nitrate, zirconium acetate, zirconium carbonate, zirconium stearate, zirconium octylate, zirconium silicate, zirconium oxynitrate, potassium zirconium carbonate, ammonium zirconium carbonate; polyvalent aldehyde compounds such as glyoxal, glutaraldehyde, aldehyde starch; methylol melamine, melamine formaldehyde resin, melamine urea resin, polyamine epichlorohydrin resin, polyamide epichlorohydrin resin, potassium persulfate, ammonium persulfate, sodium persulfate, ferric chloride, magnesium chloride, borax, boric acid, alums (aluminum potassium sulfate), ammonium chloride, and the like may be cited.

As the slipping agent used in the present invention, fatty acid metal salts such as zinc stearate, calcium stearate, and the like; waxes; silicone resins; and the like may be cited.

Stabilizing agents that improve oil resistance of recorded images and the like, such as 4,4′-butylidene (6-t-butyl-3-methylphenol), 2,2′-di-t-butyl-5,5′-dimethyl-4,4′-sulfonyl diphenol, 1,1,3-tris (2-methyl-4-hydroxy-5-cyclohexylphenyl) butane, 1,1,3-tris (2-methyl-4-hydroxy-5-t-butylphenyl) butane and the like may also be added in the range that does not adversely affect the desired effects for the problems described above.

In addition, a benzophenone type and triazole type UV absorbers, dispersion agent, de-foaming agent, antioxidant, fluorescent dye and the like may also be used.

The types and amounts of the leuco dye, color developing agent, sensitizer and other various ingredients used in the thermosensitive recording medium of the present invention may be determined according to the required performance and printability. Although the amounts of the color developing agent, the sensitizer, the pigment, the stabilizing agent and the other ingredients are not particularly restricted, from 0.5 parts to 10 parts of the color developing agent, from 0.1 parts to 10 parts of the sensitizer, from 0.5 parts to 20 parts of the pigment, from 0.01 parts to 10 parts of the stabilizing agent and from 0.01 parts to 10 parts of the other ingredients are ordinarily used per 1 part of the leuco dye.

The leuco dye, the color developing agent and the other materials added as needed are finely ground into particles with several microns or smaller in size, by using a grinder or a suitable emulsification device such as a ball mill, attritor, sand grinder and the like. The coating solutions are prepared by adding a binder and various additives to these depending on the objective. Water, alcohol and the like can be used as the solvent for the coating solution and the solid content of the coating solution is about from 20 to 40 weight %.

The thermosensitive recording medium of the present invention may further have a protective layer on the thermosensitive recording layer.

The protective layer comprises mainly a binder and a pigment, and may further comprise a crosslinking agent.

Any binder that can be used in the thermosensitive recording layer described above can be used as the binder, but carboxy-modified polyvinyl alcohol and non-core-shell type acrylic resin are preferably used. These binders may be used solely or in combination of two or more.

Any cross-linking agent that can be used in the thermosensitive recording layer described above can be used as the cross-linking agent, and epichlorohydrin-based resin and polyamine/polyamide-based resin (excluding those categorized as epichlorohydrin-based resin) are preferably used.

It is more preferable that the protective layer contains an epichlorohydrin-based resin and a polyamine/polyamide-based resin together with a carboxy-modified polyvinyl alcohol, which further improves the color developing property.

The carboxy-modified polyvinyl alcohol can be obtained as, for example, reaction product of polyvinyl alcohol and polyvalent carboxylic acid such as fumaric acid, phthalic anhydride, mellitic anhydride, itaconic anhydride etc.; esterified product of these reactants; or saponification product of copolymer of vinyl acetate and ethylenically unsaturated dicarboxylic acid such as maleic acid, fumaric acid, itaconic acid, crotonic acid, acrylic acid, methacrylic acid etc.

Specific manufacturing methods include, for example, the methods described in Japanese Patent Application Public Disclosure No. S53-91995 etc. The degree of saponification of the carboxy-modified polyvinyl alcohol is preferably 72 to 100 mol %, and the degree of polymerization is 500 to 2400, more preferably 1000 to 2000.

The glass transition point (Tg) of the non-core shell type acrylic resin is preferably 95 degree C. or lower, more preferably higher than 50 degree C. Tg is measured by differential scanning calorimetry (DSC).

The non-core-shell type acrylic resin comprises (meth)acrylic acid and a monomer component that is copolymerizable with (meth)acrylic acid. And the content of (meth)acrylic acid in the non-core-shell type acrylic resin is 1 to 10 parts by weight per 100 parts by weight of the non-core-shell type acrylic resin. (Meth)acrylic acid is alkali-soluble and has the property of making the non-core-shell type acrylic resin water-soluble by adding a neutralizing agent. By changing the non-core shell type acrylic resin to a water-soluble resin, the binding property of the resin to pigment is remarkably improved, particularly when the protective layer contains a pigment, then protective layer with excellent strength can be formed even when a large amount of pigment is contained. Examples of the component that is copolymerizable with (meth)acrylic acid include alkyl acrylate resins such as methyl (meth)acrylate, ethyl (meth)acrylate, propyl (meth)acrylate, butyl (meth)acrylate, isobutyl (meth)acrylate, pentyl (meth)acrylate, hexyl (meth)acrylate, 2-ethylhexyl (meth)acrylate and octyl (meth)acrylate etc., a modified alkyl acrylate resin such as the above alkyl acrylate resin modified with epoxy resin, silicone resin, styrene or derivative thereof, (meth)acrylonitrile, acrylic acid ester, and hydroxyalkyl acrylic acid ester. In particular, (meth)acrylonitrile and/or methyl methacrylate are preferable. And the content of (meth)acrylonitrile in the non-core-shell type acrylic resin is 15 to 70 parts by weight per 100 parts by weight of the non-core-shell type acrylic resin. Further, the content of methyl methacrylate in the non-core-shell type acrylic resin is preferably 20 to 80 parts by weight per 100 parts by weight of the non-core-shell type acrylic resin. In the case of containing (meth)acrylonitrile and methyl methacrylate, the content of (meth)acrylonitrile in the non-core-shell type acrylic resin is preferably 15 to 18 parts by weight per 100 and the content of methyl methacrylate in the non-core-shell type acrylic resin is preferably 20 to 80 parts by weight per 100 parts by weight of the non-core-shell type acrylic resin.

The epichlorohydrin-based resin is a resin characterized by containing an epoxy group in the molecule and include, for example, polyamide epichlorohydrin resin, polyamine epichlorohydrin resin and the like. These may be used solely or in combination. As the amine existing in the main chain of the epichlorohydrin-based resin, from primary amine to quaternary amines can be used and is not particularly limited. Furthermore, the cationization degree of the resin is preferably 5 cationization degree or less (measured at pH 7) and the molecular weight of the resin is preferably 500,000 or more, since the resin is superior in water resistance. Specific examples of the epichlorohydrin-based resin include Sumirez resin 650 (30), Sumirez resin 675A, Sumirez resin 6615 (Sumitomo Chemical Co., Ltd., Japan), WS4002, WS4020, WS4024, WS4030, WS4046, WS4010, CP8970 (Seiko PMC Corporation, Japan) and the like.

The polyamine/polyamide resin does not have an epoxy group in the molecule and include, for example, polyamide urea resin, polyalkylene polyamine resin, polyalkylene polyamide resin, polyamine polyurea resin, modified polyamine resin, modified polyamide resin, polyalkylene polyamine urea formalin resin, polyalkylene polyamine polyamide polyurea resin and the like. These may be used solely or in combination.

Specific examples of the polyamine/polyamide resin include: Sumirez resin 302 (polyamine polyurea resin manufactured by Sumitomo Chemical Co., Ltd.), Sumirez resin 712 (polyamine polyurea resin manufactured by Sumitomo Chemical Co., Ltd.), Sumirez resin 703 (polyamine polyurea resin manufactured by Sumitomo Chemical Co., Ltd.), Sumirez resin 636 (polyamine polyurea resin manufactured by Sumitomo Chemical Co., Ltd.), Sumirez resin SPI-100 (modified polyamine resin manufactured by Sumitomo Chemical Co., Ltd.), Sumirez resin SPI-102A (modified polyamine resin manufactured by Sumitomo Chemical Co., Ltd.), Sumirez resin SPI-106N (modified polyamide resin manufactured by Sumitomo Chemical Co., Ltd.), Sumirez resin SPI-203 (50) (Sumitomo Chemical Co., Ltd.), Sumirez Resin SPI-198 (Sumitomo Chemical Co., Ltd.), PRINTIVE A-700, PRINTIVE A-600 (Asahi Kasei Corp.), PA6500, PA6504, PA6634, PA6638, PA6640, PA6644, PA6646, PA6654, PA6702, PA6704 (polyalkylene polyamine polyamide polyurea resin manufactured by Seiko PMC Corporation), CP 8994 (polyethyleneimine resin manufactured by Seiko PMC Corporation) and the like. Polyamine resin (polyalkylene polyamine resin, polyamine polyurea resin, modified polyamine resin, polyalkylene polyamine urea formalin resin, polyalkylene polyamine polyamide polyurea resin) is preferably used since these print intensities are better, but is not limited in particular.

The content of epichlorohydrin-based resin and the polyamine/polyamide-based resin in the protective layer are preferably 1 to 100 parts by weight, more preferably 5 to 50 parts by weight, further preferably 10 to 40 parts by weight, each per 100 parts by weight of the carboxy-modified polyvinyl alcohol, when these are used together with the carboxy-modified polyvinyl alcohol, in the protective layer.

Any pigment usable in the thermosensitive recording layer may be used in the protective layer. As the pigment, for example, kaolin, calcined kaolin, aluminum hydroxide, silica are preferable. These pigments may be used solely or in combination of two or more.

The content (in solid) of the pigments in the protective layer is preferably 20 weight % or more, more preferably from 20 to 80 weight %. In the case that the protective layer contains pigments, the total content (in solid) of the pigment and the binder is preferably about 30 to 300 parts by weight per 100 parts by weight of the pigment.

The coating solution for the protective layer may contain, if necessary, various additives such as a crosslinking agent, a lubricant, a stabilizer, an ultraviolet absorber, a dispersant, a defoaming agent, an antioxidant, a fluorescent dye, etc. that can be used for the thermosensitive recording layer.

The thermosensitive recording medium of the present invention may further have an undercoat layer between the support and the thermosensitive recording layer.

The undercoat layer comprises mainly a binder and a pigment. As the binder used for the undercoat layer, the binders shown above as the materials that can be used for the thermosensitive recording layer may be used. These binders may be used alone or in combination of two or more.

Any generally used pigment may be used as the pigment in the undercoat layer. As the pigment, for example, inorganic pigment, such as calcium carbonate, silica, zinc oxide, titanium oxide, aluminum hydroxide, magnesium hydroxide, calcined kaolin, clay, talc and the like and organic pigments such as hollow plastic particles may be used. These pigments may be used alone or in combination of two or more.

The hollow rate by volume of the hollow plastic particles is preferably about 40 to 95%. By setting the hollow rate by volume to 40% or more, the heat insulating property can be improved and the color developing property can be further enhanced. On the other hand, when the hollow rate by volume is 95% or less, the strength of the shell of the hollow particles is increased, the hollow state is effectively maintained, and it becomes easy to obtain an undercoat layer having good surface strength. Here, the hollow rate by volume is a value obtained by the formula (d3/D3)×100, in which “d” represents the inner diameter of the organic hollow particles and “D” represents the outer diameter of the organic hollow particles.

The amount of the pigments in the undercoat layer is ordinarily from 50 to 95 weight parts, preferably from 70 to 90 weight parts per 100 parts by weight of the total solid of the undercoat layer.

The amount of the pigment in the undercoat layer is usually 50 to 95 parts by weight, preferably 70 to 90 parts by weight, based on 100 parts by weight of the total solid content. In the present invention, the undercoat layer preferably contains the hollow plastic particles as a pigment. When the undercoat layer contains the hollow plastic particles as a pigment, the content of the hollow plastic particles is preferably 15% by weight or more, more preferably 45% by weight or more, based on the total amount (solid content) of the pigment. The abovementioned inorganic pigments may be used as other pigment than the hollow plastic particles in the undercoat layer, and it is preferable to use calcined kaolin.

Various aids such as a dispersion agent, plasticizer, pH controlling agent, de-foaming agent, water retention agent, preservative, coloring dye, UV absorber and the like may be added to the undercoat layer, as required.

In the present invention, the method for coating the thermosensitive recording layer and other coating layers is not limited in particular, but any known conventional techniques may be used. The method for coating may be appropriately selected from off-machine coating machines and on-machine coating machines, which are equipped with coaters such as air knife coater, rod blade coater, bent blade coater, bevel blade coater, roll coater, curtain coater and the like.

The coating amounts of the thermosensitive recording layer and other coating layers are not limited in particular, but may be determined according to the required performance and the recording suitability.

The typical coating amount (in solid) of the thermosensitive recording layer is ordinarily in the range of from 2 to 12 g/m², the typical coating amount (in solid) of the undercoat layer is ordinarily in the range of from 1 to 15 g/m² and the typical coating amount (in solid) of the protective layer is ordinarily in the range of from 1 to 5 g/m², preferably from 1 to 3 g/m².

Furthermore, various technologies known in the thermosensitive recording medium field, such as a flattening treatment such as super calendaring and the like can be applied as needed after coating individual coating layers.

### Examples

The following Examples illustrate the present invention, but the Examples are not intended to limit the scope of the present invention. In the following description, the terms parts and % indicate parts by weight and weight %, respectively.

The coating solutions and dispersions were prepared as described below.

### [Preparation of Coating Solutions]

Undercoat layer coating solution was prepared by dispersing and stirring the following formulation:

### Undercoat Layer Coating Solution

| | |
|---|---|
| Calcined kaolin (BASF Co.: Ansilex 90) | 100.0 parts |
| Styrene-butadiene copolymer latex (Zeon | 10.0 parts |
| Corporation, ST5526, solid content: 48%) | |
| Water | 50.0 parts |

Color developing agent dispersions (Solutions A1 to A6), a leuco dye dispersion (Solution B) and a sensitizer dispersion (Solution C) with the following formulations were separately wet ground using sand grinders until the average particle sizes were about 0.5 μm.

### Color Developing Agent Dispersion 1 (Solution A1)

| | |
|---|---|
| 4-hydroxy-4′-benzyloxy-diphenylsulfone | 6.0 parts |
| (Nicca Chemical Co., Ltd., BPS-MA3) | |
| Aqueous solution of completely saponified | 5.0 parts |
| polyvinyl alcohol (Kuraray Co., Ltd., | |
| PVA117, solid content: 10%) | |
| Water | 1.5 parts |

### Color Developing Agent Dispersion 2 (Solution A2)

| | |
|---|---|
| Phenolic compound represented by the formula 7 | 6.0 parts |
| (Nippon Soda Co., Ltd., D-90) | |
| Aqueous solution of completely saponified | 5.0 parts |
| polyvinyl alcohol (PVA117) | |
| Water | 1.5 parts |

### Color Developing Agent Dispersion 3 (Solution A3)

| | |
|---|---|
| Urea-urethane-based compound represented by the | 6.0 parts |
| formula 8 (Chemipro Kasei Kaisha, LTD., UU) | |
| Aqueous solution of completely saponified | 5.0 parts |
| polyvinyl alcohol (PVA117) | |
| Water | 1.5 parts |

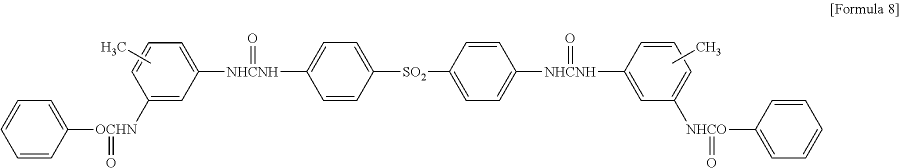

### Color Developing Agent Dispersion 4 (Solution A4)

| | |
|---|---|
| 4-hydroxy-4′-n-propoxy-diphenylsulfone | 6.0 parts |
| (Mitsubishi Chemical Corporation, TOMILAC KN) | |
| Aqueous solution of completely saponified | 5.0 parts |
| polyvinyl alcohol (PVA117) | |
| Water | 1.5 parts |

### Color Developing Agent Dispersion 5 (Solution A5)

| | |
|---|---|
| 4-hydoroxypheny-4′-phenoxyphenylsulfone | 6.0 parts |
| (represented by formula 9) | |
| Aqueous solution of completely saponified | 5.0 parts |
| polyvinyl alcohol (PVA117) | |
| Water | 1.5 parts |

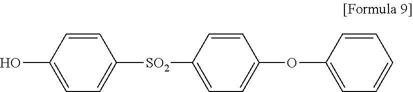

### Color Developing Agent Dispersion 6 (Solution A6)

| | |
|---|---|
| 1-[4-(4-hydroxyphenylsulfonyl) phenoxy]-4-[4- | 6.0 parts |
| (4-isopropoxyphenylsulfonyl) phenoxy] butane | |
| (Mitsubishi Chemical Corporation, TOMILAC 214) | |
| Aqueous solution of completely saponified | 5.0 parts |
| polyvinyl alcohol (PVA117) | |
| Water | 1.5 parts |

### Leuco Dye Dispersion (Solution B)

| | |
|---|---|
| 3-Dibutylamino-6-methyl-7-anilinofluorane | 6.0 parts |
| (Yamamoto Chemicals Inc., ODB-2) | |
| Aqueous solution of completely saponified | 5.0 parts |
| polyvinyl alcohol (PVA117) | |
| Water | 1.5 parts |

### Sensitizer Dispersion (Solution C)

| | |
|---|---|
| 1,2-bis(2-Methylphenoxy) ethane (Sanko Co. | 6.0 parts |
| Ltd, KS232) | |
| Aqueous solution of completely saponified | 5.0 parts |
| polyvinyl alcohol (PVA117) | |
| Water | 1.5 parts |

Next, these dispersions were blended in the proportion described below to prepare the thermosensitive recording layer coating solutions 1 and 2.

### Thermosensitive Recording Layer Coating Solution 1

| | |
|---|---|
| Color developing agent dispersion (Solution A1) | 18.0 parts |
| Color developing agent dispersion (Solution A2) | 18.0 parts |
| Leuco dye dispersion (Solution B) | 18.0 parts |
| Sensitizer dispersion (Solution C) | 9.0 parts |
| Silica dispersion (Mizusawa Industrial Chemicals, | 17.5 parts |
| Ltd., Mizukasil P-537, solid content: 25%) | |
| Aqueous solution of completely saponified | 25.0 parts |
| polyvinyl alcohol (PVA117) | |

### Thermosensitive Recording Layer Coating Solution 2

| | | |
|---|---|---|
| Color developing agent dispersion (Solution A1) | 18.0 | parts |
| Color developing agent dispersion (Solution A2) | 18.0 | parts |
| Leuco dye dispersion (Solution B) | 18.0 | parts |
| Sensitizer dispersion (Solution C) | 9.0 | parts |
| Silica dispersion (Mizukasil P-537) | 7.5 | parts |
| Aqueous solution of completely saponified | 25.0 | parts |
| polyvinyl alcohol (PVA117) | | |

Next, protective layer coating solution was prepared by mixing the following formulations:

### Protective Layer Coating Solution

| | |
|---|---|
| Aluminum hydroxide dispersion (Martinsberg: | 9.0 parts |
| Martifin OL, solid content: 50%) | |
| Carboxyl modified polyvinyl alcohol solution | 30.0 parts |
| (Kuraray Co., Ltd,: KL318, solid content: 10%) | |
| Polyamide epichlorohydrin resin (Seiko PMC: | 4.0 parts |
| WS4030, solid content: 25%) | |
| Zinc stearate dispersion (Chukyo Yushi Co., | 2.0 parts |
| Ltd.: HydrinZ-7-30, solid content: 30%) | |

### Example 1

The undercoat layer coating solution was applied on one side of a support (groundwood free paper with a basis weight of 47 g/m²) by using a bent blade coater with a coating amount (in solid) of 10.0 g/m², and was dried to prepare an undercoated paper.

The thermosensitive recording layer coating solution 1 was applied on the undercoat layer of the undercoated paper by using a rod blade coater with a coating amount (in solid) of 6.0 g/m² and was dried and super calendared so that the smoothness was 500-1,000 seconds to prepare a thermosensitive recording medium.

### Example 2

A thermosensitive recording medium was prepared in the same manner as described in Example 1 using the thermosensitive recording layer coating solution 1 with the exception of changing the amount of the color developing agent dispersion (Solution A1) to 31.5 parts, and changing the amount of the color developing agent dispersion (Solution A2) to 4.5 parts.

### Example 3

A thermosensitive recording medium was prepared in the same manner as described in Example 1 using the thermosensitive recording layer coating solution 1 with the exception of changing the amount of the color developing agent dispersion (Solution A1) to 25.5 parts, and changing the amount of the color developing agent dispersion (Solution A2) to 10.5 parts.

### Example 4

A thermosensitive recording medium was prepared in the same manner as described in Example 1 using the thermosensitive recording layer coating solution 1 with the exception of changing the amount of the color developing agent dispersion (Solution A1) from 18.0 parts to 9.0 parts and adding 9.0 parts of the color developing agent dispersion (Solution A6).

### Example 5

A thermosensitive recording medium was prepared in the same manner as described in Example 1 using the thermosensitive recording layer coating solution 1 with the exception of changing the amount of the color developing agent dispersion (Solution A2) from 18.0 parts to 8.0 parts and adding 10.0 parts of the color developing agent dispersion (Solution A3).

### Example 6

The undercoat layer coating solution was applied on one side of a support (groundwood free paper with a basis weight of 47 g/m²) by using a bent blade coater with a coating amount (in solid) of 10.0 g/m², and was dried to prepare an undercoated paper.

The thermosensitive recording layer coating solution 2 was applied on the undercoat layer of the undercoated paper by using a rod blade coater with a coating amount (in solid) of 6.0 g/m² and was dried and super calendared so that the smoothness was 500-1,000 seconds to prepare a thermosensitive recording medium.

Then the protective layer coating solution was applied on the thermosensitive recording layer of the thermosensitive recording layer coated paper by using a rod blade coater with a coating amount (in solid) of 2.0 g/m² and was dried to prepare a thermosensitive recording medium.

### Comparative Example 1

A thermosensitive recording medium was prepared in the same manner as described in Example 1 using the thermosensitive recording layer coating solution 1 with the exception of changing the amount of the color developing agent dispersion (Solution A1) from 18.0 parts to 36.0 parts and excluding the color developing agent dispersion (Solution A2).

### Comparative Example 2

A thermosensitive recording medium was prepared in the same manner as described in Example 1 using the thermosensitive recording layer coating solution 1 with the exception of changing the amount of the color developing agent dispersion (Solution A2) from 18.0 parts to 36.0 parts and excluding the color developing agent dispersion (Solution A).

### Comparative Example 3

A thermosensitive recording medium was prepared in the same manner as described in Example 1 using the thermosensitive recording layer coating solution 1 with the exception of using the color developing agent dispersion (Solution A3) in place of the color developing agent dispersion (Solution A2).

### Comparative Example 4

A thermosensitive recording medium was prepared in the same manner as described in Example 1 using the thermosensitive recording layer coating solution 1 with the exception of using the color developing agent dispersion (Solution A4) in place of the color developing agent dispersion (Solution A1).

### Comparative Example 5

A thermosensitive recording medium was prepared in the same manner as described in Example 1 using the thermosensitive recording layer coating solution 1 with the exception of using 4.5 parts of the color developing agent dispersion (Solution A1) and 31.5 parts of the color developing agent dispersion (Solution A2).

### Comparative Example 6

A thermosensitive recording medium was prepared in the same manner as described in Example 1 using the thermosensitive recording layer coating solution 1 with the exception of using the color developing agent dispersion (Solution A5) in place of the color developing agent dispersion (Solution A1).

The prepared thermosensitive recording media were evaluated as below.

### <Color Developing Property (Recorded Density)>

A checkerboard pattern was painted on the prepared thermosensitive recording media by using a thermosensitive recording medium print tester (Okura Engineering Co., Ltd. TH-PMD equipped with a thermal head by Kyocera Co.) at applied energy of 0.35 mJ/dot and printing speed of 50 mm/sec. The density of the printed portion was measured by using Macbeth Densitometer (RD-914, with Amber filter) to evaluate the color developing property (recorded density).

### <Bar Code Readability>

The prepared thermosensitive recording medium was treated in the following two environments for 24 hours and stored in an environment of 23 degree C., 50% RH for three hours.

(1) 80 degree C.

(2) 50 degree C., 90% RH

Then a bar code (CODE39) was printed on the thermosensitive recording medium by using a label printer (140XiII manufactured by Zebra Co., Ltd.) at print level of plus 10 and print speed of 15.2 cm per sec. (6 inches per sec.). Then the printed bar code was read by a bar code tester (Honeywell, QCPC600, light source: 640 nm). The bar code readability was evaluated according to the symbol grades of the ANSI standard as in the manner described below.

Symbol Grade: The bar code is divided into ten pieces in the direction vertical to the bar. The reading test is conducted once each to average the results. And the averaged bar code readability is rated as below:

Rating: (Excellent) A>B>C>D>F (Poor)

<Discoloration (Severe Environment (1): Long-Term Storage Under High Temperature and/or High Humidity Conditions)>

The prepared thermosensitive recording medium was stored at 50 degree C. and 95% RH for 4 days and then left standing at 23 degree C. and 50% RH for 3 hours.

The color density of non-printed portion (i.e. blank portion) was measured by using Macbeth Densitometer (RD-914, with Amber filter) and the background color value was calculated from the difference between the color densities before and after the treatment. The heat discoloration resistance in the blank portion was evaluated on the following criteria.

Background color value=(color density of the non-printing portion after the treatment)−(color density of the non-printing portion before the treatment)

Excellent: The background color value is less than 0.1

Good: The background color value is 0.1 or higher and less than 0.3

Fair: The background color value is 0.3 or higher and less than 0.5

Poor: The background color value is 0.5 or higher

### <Discoloration (Severe Environment (2): Heating by Microwave Oven)>

The prepared thermosensitive recording medium was printed with a label printer 140XiIII manufactured by Zebra Co., Ltd. at a printing level of +10 and a printing speed of 15.2 cm/sec (6 inches/sec), and then heat-treated in a microwave oven with an output of 800 W for 2 minutes, and then left standing at 23 degree C. and 50/RH for 3 hours. The discoloration was evaluated by visual inspection with the following criteria:
Excellent: The non-printed portion (i.e. blank portion) is not colored (i.e. discolored), and characters can be read without problems.
Good: The non-printed portion is slightly colored, but characters can be read.
Fair: The non-printed portion is colored, but characters can be read.
Poor: The non-printed portion markedly colored and the characters cannot be read.

### <Plasticizer Resistance>

Checkerboard pattern was printed on the prepared thermosensitive recording media by using a printing tester for thermosensitive recording paper (Okura Engineering Co. LTD., TH-PMD equipped with a thermal head by Kyocera Corporation) at recording energy of 0.35 mJ/dot and recording speed of 50 mm/sec. A paper tube was wrapped once with polyvinyl chloride wrap (Mitsui Toatsu Chemical: High Wrap KMA) and the thermosensitive recording medium was placed on the wrapped paper tube so that the recorded face is the outer face. Furthermore, the tube was wrapped 3 times with polyvinyl chloride wrap and was left standing for 24 hours under the following environmental conditions.

(1) 23 degree C., 50% RH

(2) 50 degree C., 50% RH

The record density of the recorded section was measured by using Macbeth densitometer (RD-914, with amber filter), and the residual ratio was calculated from the measured value before and after the treatment according to the following equation:

Residual ratio (%)=(record density after the treatment/record density before the treatment)×100

The plasticizer resistance was evaluated by the following criteria from the obtained residual value:
Excellent: The residual rate is 90% or more
Good: The residual rate is 75% or more and less than 90
Fair: The residual rate is 50% or more and less than 75%
Poor: The residual rate is less than 50%

If the evaluation is Excellent or Good, there is no practical problem.

The evaluation results are shown in Table 1.

TABLE 1

| | | | | Color developing | | |
|---|---|---|---|---|---|---|
| | | | | agent 2/Color | | |
| | Color developing agent | | | developing | Protective | Recorded |
| | 1 | 2 | 3 | agent 1 | layer | density |
| Example1 | BPS-MA3 | D90 | — | 1.0 | — | 1.33 |
| Example2 | BPS-MA3 | D90 | — | 0.14 | — | 1.35 |
| Example3 | BPS-MA3 | D90 | — | 0.41 | — | 1.34 |
| Example4 | BPS-MA3 | D90 | TOMILAC214 | 2.0 | — | 1.30 |
| Example5 | BPS-MA3 | D90 | UU | 0.44 | — | 1.32 |
| Example6 | BPS-MA3 | D90 | — | 1.0 | installed | 1.28 |
| Comparative | BPS-MA3 | — | — | — | — | 1.35 |
| Example1 | | | | | | |
| Comparative | — | D90 | — | — | — | 0.97 |
| Example2 | | | | | | |
| Comparative | BPS-MA3 | UU | — | 1.0 | — | 1.30 |
| Example3 | | | | | | |
| Comparative | TOMILAC KN | D90 | — | 1.0 | — | 1.27 |
| Example4 | | | | | | |
| Comparative | BPS-MA3 | D90 | — | 7.0 | — | 1.24 |
| Example5 | | | | | | |
| Comparative | ** | D90 | — | 1.0 | — | 1.22 |
| Example6 | | | | | | |

| | Bar code readability | | Discoloration | | Plasticizer resistance | |
|---|---|---|---|---|---|---|
| | (1) | (2) | (1) | (2) | (1) | (2) |
| Example1 | B | B | Good | Excellent | Good | Good |
| Example2 | B | B | Excellent | Good | Good | Good |
| Example3 | B | B | Good | Excellent | Good | Good |
| Example4 | B | B | Excellent | Excellent | Good | Good |
| Example5 | B | B | Excellent | Excellent | Excellent | Excellent |
| Example6 | B | B | Excellent | Excellent | Excellent | Good |
| Comparative | C | D | Excellent | Poor | Poor | Poor |
| Example1 | | | | | | |
| Comparative | F | F | Good | Poor | Good | Fair |
| Example2 | | | | | | |
| Comparative | B | B | Poor | Fair | Poor | Poor |
| Example3 | | | | | | |
| Comparative | C | D | Fair | Poor | Fair | Poor |
| Example4 | | | | | | |
| Comparative | C | C | Poor | Fair | Good | Fair |
| Example5 | | | | | | |
| Comparative | F | F | Good | Fair | Good | Fair |
| Example6 | | | | | | |

** 4-hydoroxypheny-4′-phenoxyphenylsulfone

From Table 1, when the above-mentioned sulfone compound and the above-mentioned phenolic compound are contained in the thermosensitive recording layer as a color developing agent in a specific ratio, the color developing property under severe environment, particularly barcode readability, is excellent and the thermosensitive recording layer was found resistant to discoloration under severe environment. Furthermore, when the thermosensitive recording layer further contains a urea-urethane-based compound as a color developing agent, the plasticizer resistance was remarkably improved.

1. A thermosensitive recording medium having a thermosensitive recording layer comprising a colorless or pale colored electron donating leuco dye and an electron accepting color developing agent on a substrate, wherein the thermosensitive recording layer comprises a sulfone compound and a phenolic compound as the electron accepting color developing agent, wherein the sulfone compound is represented by the general formula 1 and the phenolic compound is a compound containing two or more hydroxyl groups and two or more diphenylsulfone backbone and containing neither urethane bond nor urea bond, wherein the content of the phenolic compound is from 0.01 to 1.0 parts by weight per 1 part by weight of the sulfone compound. 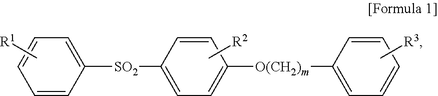
wherein R¹ represents a hydrogen atom or a hydroxyl group, R² and R³, which may be identical to or different from the others, represent a hydrogen atom or an alkyl group or an alkoxy group having 1-6 carbon atoms, and m represents an integer of 1 to 3.

2. The thermosensitive recording medium of claim 1, wherein the thermosensitive recording layer further comprises, as the electron accepting color developing agent, a urea-urethane-based compound represented by the following general formula:

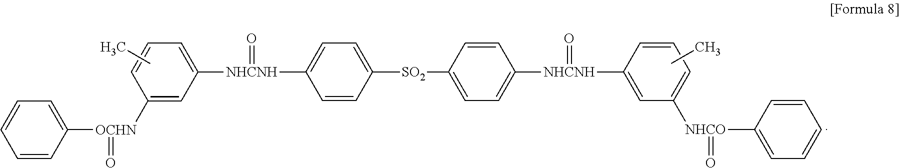

3. The thermosensitive recording medium of claim 2, wherein the content of the urea-urethane-based compound is from 0.01 to 1.0 parts by weight per 1 part by weight of the sulfone compound.

4. The thermosensitive recording medium of claim 1, wherein the phenolic compound is represented by the following general formula: 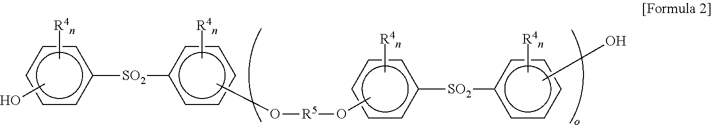
wherein R⁴, which may be identical or different, represents a halogen atom or an alkyl group or an alkenyl group having 1 to 6 carbon atoms,
n, which may be identical or different, represents an integer of from 0 to 4,
o is an integer of from 1 to 11,
R⁵, which may be identical or different, is a hydrocarbon group having 1 to 12, which may have an ether bond, which may be saturated or unsaturated, which may be linear or branched, a substituted phenylene group represented by a general formula below: 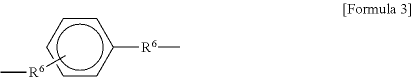
in the formula, R⁶ represents a methylene group or an ethylene group, or a divalent group represented by a general formula below: 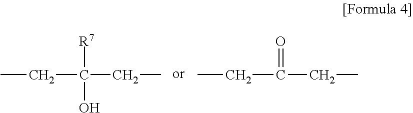
in the formula, R⁷ represents a hydrogen atom or an alkyl group having 1 to 4 carbon atoms.

5. The thermosensitive recording medium of claim 1, wherein the content of the phenolic compound is from 0.05 to 0.8 parts by weight per 1 part by weight of the sulfone compound.

6. The thermosensitive recording medium of claim 1, wherein the sulfone compound is represented by the general formula: 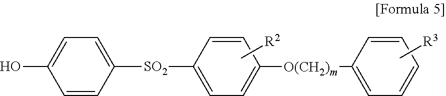
wherein R² and R³ are as defined above, and m represents 1 or 2.

7. The thermosensitive recording medium of claim 1, wherein at least one of R² and R³ represents a hydrogen atom.

8. The thermosensitive recording medium of claim 1, wherein m represents 1.

9. The thermosensitive recording medium of claim 1, wherein the phenolic compound is represented by the following formula:

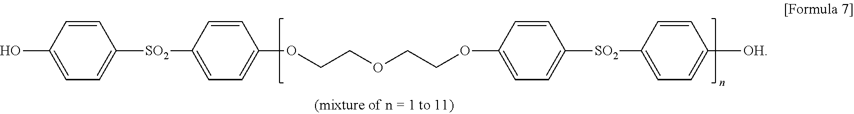

10. The thermosensitive recording medium of claim 2, wherein the phenolic compound is represented by the following general formula: 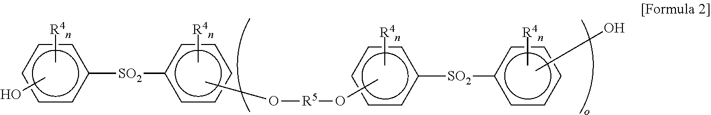
wherein R⁴, which may be identical or different, represents a halogen atom or an alkyl group or an alkenyl group having 1 to 6 carbon atoms,
n, which may be identical or different, represents an integer of from 0 to 4,
o is an integer of from 1 to 11,
R⁵, which may be identical or different, is a hydrocarbon group having 1 to 12, which may have an ether bond, which may be saturated or unsaturated, which may be linear or branched, a substituted phenylene group represented by a general formula below: 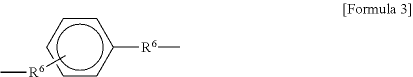
in the formula, R⁶ represents a methylene group or an ethylene group, or
a divalent group represented by a general formula below: 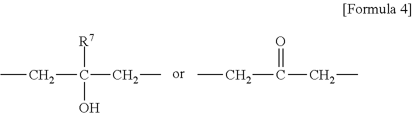
in the formula, R⁷ represents a hydrogen atom or an alkyl group having 1 to 4 carbon atoms.

11. The thermosensitive recording medium of claim 3, wherein the phenolic compound is represented by the following general formula: 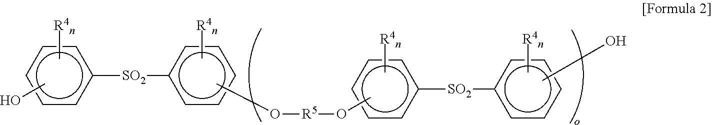
wherein R⁴, which may be identical or different, represents a halogen atom or an alkyl group or an alkenyl group having 1 to 6 carbon atoms,
n, which may be identical or different, represents an integer of from 0 to 4,
o is an integer of from 1 to 11,
R⁵, which may be identical or different, is a hydrocarbon group having 1 to 12, which may have an ether bond, which may be saturated or unsaturated, which may be linear or branched, a substituted phenylene group represented by a general formula below: 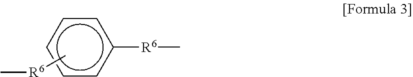
in the formula, R⁶ represents a methylene group or an ethylene group, or
a divalent group represented by a general formula below: 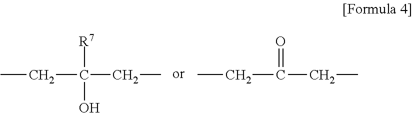
in the formula, R⁷ represents a hydrogen atom or an alkyl group having 1 to 4 carbon atoms.

12. The thermosensitive recording medium of claim 2, wherein the content of the phenolic compound is from 0.05 to 0.8 parts by weight per 1 part by weight of the sulfone compound.

13. The thermosensitive recording medium of claim 3, wherein the content of the phenolic compound is from 0.05 to 0.8 parts by weight per 1 part by weight of the sulfone compound.

14. The thermosensitive recording medium of claim 4, wherein the content of the phenolic compound is from 0.05 to 0.8 parts by weight per 1 part by weight of the sulfone compound.

15. The thermosensitive recording medium of claim 2, wherein the sulfone compound is represented by the general formula: 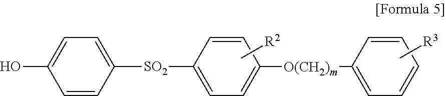
wherein R² and R³ are as defined above, and m represents 1 or 2.

16. The thermosensitive recording medium of claim 3, wherein the sulfone compound is represented by the general formula: 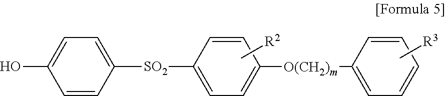
wherein R² and R³ are as defined above, and m represents 1 or 2.

17. The thermosensitive recording medium of claim 4, wherein the sulfone compound is represented by the general formula: 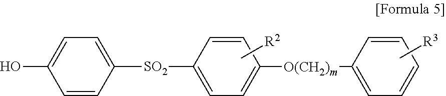
wherein R² and R³ are as defined above, and m represents 1 or 2.

18. The thermosensitive recording medium of claim 5, wherein the sulfone compound is represented by the general formula: 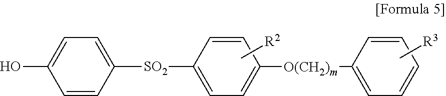
wherein R² and R³ are as defined above, and m represents 1 or 2.

19. The thermosensitive recording medium of claim 2, wherein at least one of R² and R³ represents a hydrogen atom.

20. The thermosensitive recording medium of claim 2, wherein m represents 1.
